(19) United States
(12) Patent Application Publication
(10) Pub. No.: US 2016/0067100 A1
(43) Pub. Date: Mar. 10, 2016

(54) AURAL FOREIGN BODY REMOVAL DEVICE AND RELATED METHODS OF USE AND MANUFACTURE

(71) Applicants: Shayn Peirce COTTLER, Charlottesville, VA (US); Bradley W. KESSER, Charlottesville (US); Meg G. KEELEY, Richmond, VA (US); Leigh AMATEAU, Richmond, VA (US); UNIVERSITY OF VIRGINIA PATENT FOUNDATION, Charlottesville, VA (US)

(72) Inventors: Shayn Peirce Cottler, Charlottesville, VA (US); Bradley W. Kesser, Charlottesville, VA (US); Meg G. Keeley, Charlottesville, VA (US); Leigh Amateau, Richmond, VA (US)

(21) Appl. No.: 14/890,879

(22) PCT Filed: May 15, 2014
(86) PCT No.: PCT/US14/38024

§ 371 Date: Nov. 12, 2015

Related U.S. Application Data
(60) Provisional application No. 61/823,496, filed on May 15, 2013.

(51) Int. Cl. A61F 11/00

(52) U.S. Cl. CPC A61F 11/006

(57) ABSTRACT

A device and related method of use for removing a foreign object from the external ear canal or the nasal cavity is provided. The device comprises an elongated member having a first portion, a second portion with a distal end, and a fulcrum disposed along the elongated member between the first and the second portions.

### CROSS-REFERENCE TO RELATED APPLICATIONS

The present application claims benefit of priority under 35 U.S.C. §119(e) from U.S. Provisional Application Ser. No. 61/823,496, filed May 15, 2013, entitled “Aural Foreign Body Removal Device and Related Method;” the disclosure of which is hereby incorporated by reference herein in its entirety.

### TECHNICAL FIELD

This invention relates generally to the field of medical devices to be used for removing foreign objects from small anatomical spaces. More specifically, the invention is directed towards a device and methods for removing foreign objects from the external ear canal or the nasal cavity.

### BACKGROUND

Each year, physicians see thousands of children for foreign bodies located in their ear canal or nasal cavity, and in many cases these objects cannot be removed using the current standards of care. It is estimated that foreign body extraction from a child's ear canal accounts for approximately 1 in every 500 emergency room (ER) visits. See Schulze S L, Kerschner J, Beste D, Pediatric external auditory canal foreign bodies: a review of 698 cases. Otolarngol Head Nick Surg 2002; 127: 73-78. Common devices used include alligator forceps, Frazier tip suctions, cerumen loops, right angle ball hooks, and Hartman forceps. In one study, spherical objects only had a 51% successful removal rate by pediatricians. In addition, non-graspable foreign objects are also more common than graspable objects. See DiMuzio J, Deschler D, Emergency Department Management of Foreign Bodies of the External Ear Canal in Children, Otol & Neurotol 2002; 23: 473-5.

Existing devices are not only unsuitable for removing spherical bodies, but can also be dangerous to a child's ear canal. The most common methods of removing foreign bodies from the ear canal or nasal cavity are through the use of forceps (alligator and Hartman), balloon catheters, suction, and irrigation. Alligator forceps look like scissors except they contain very small pincers on the end of a thin metal rod. Hartman forceps are similar to alligator forceps, but the end has the appearance of tweezers. Both alligator forceps and Hartman forceps may be used for removing foreign bodies that are graspable or have edges, such as paper or insects; however, they cannot grip small, spherical objects. The sharp edges of the forceps can damage the ear canal or tympanic membrane, posing additional risks. See Kadish H, Ear and Nose Foreign Bodies: “It is All About the Tools,” Clin Pediatr 2005; 44: 665-70. Another less commonly used device is the Katz extractor that works similar to a catheter. First, a deflated balloon is inserted into the ear canal so that the tip extends beyond the foreign object. The physician then depresses a bulb in order to inflate the balloon tip. The balloon is then pulled to dislodge the object and guide it out from within the ear canal. U.S. Pat. No. 5,454,817 teaches a balloon catheter. This Katz method can be effective to some extent, but it will not work if the catheter tip cannot get past the foreign body, which is often the case in a child's narrow ear canal. The risk of damaging the tympanic membrane stills exists with the Katz extractor. See DiMuzio J, Deschler D, Emergency Department Management of Foreign Bodies of the External Ear Canal in Children, Otol & Neurotol 2002; 23: 473-5.

Irrigation and suction are also common removal methods. However, both methods pose danger to the sensitive tympanic membrane of a young child. Suction is useful for removing foreign bodies, especially those that are light, hard and spherical, but it loses effectiveness in the narrower, medial two thirds of the ear canal. The loud sound of the suction can also be aggravating or traumatizing to pediatric patients. Furthermore, suction is usually only operable by otolaryngologists and seldom by the ER or pediatricians. See Kadish H, Ear and Nose Foreign Bodies: “It is All About the Tools,” Clin Pediatr 2005; 44: 665-70. Irrigation of the ear canal is another possible technique to remove spherical objects that cannot be grasped by forceps or suction. However, if the object absorbs the irrigation solution, it becomes even more difficult to remove. In addition, if the irrigation pressure is too high, practitioners also run the risk of rupturing the tympanic membrane. See Kadish H, Ear and Nose Foreign Bodies: “It is All About the Tools,” Clin Pediatr 2005; 44: 665-70. The ER or the pediatricians' offices are also usually not equipped with the suction or irrigation apparatus. The patient must be referred to an otolaryngologist when a pediatrician or ER physician is unsuccessful at removing the object, and often, a surgical procedure is necessary. Overall, current standards of care are not sufficient for the removal of all foreign bodies. Despite continued efforts to develop new products in this field, this problem has not been effectively addressed.

Certain swab-like devices are used for the removal of cerumen from the ear canal. See, for example, U.S. Pat. No. 8,551,031 and U.S Patent Application 2008/0208100. However, these devices are better suited for removing cerumen, which is a waxy type of substance secreted by glandular cells in the ear canal of the human body, and do not adequately remove small spherical foreign objects, such as beads. Firstly, swab-like devices usually are designed with a dimension that prevents the user from inadvertently advancing the tip of the device to the medial ear canal. As a result, such devices will not be useful in retrieving a foreign object that is lodged at the narrowing section of external ear canal, called the bony-cartilaginous junction, or beyond the bony-cartilaginous junction to the medial two third of the ear canal, the osseous portion of the ear canal. They will also not leave any space to allow visualization of the external ear canal when the tip of the swab-like device is inside the external ear canal. Furthermore, such devices will be too large to be able to pass through a speculum and thus cannot be used in combination with a speculum or an otoscope. Secondly, the size of the swab-like device makes it virtually impossible for the swab-like device to not touch the wall of the external ear canal. While it is an objective of a swab in the prior art to contact the wall of the external ear canal to dislodge the cerumen that is stuck on the wall, it is undesirable for the adhesive on a device that is intended for removal of a foreign object to make contact with the wall of the external ear canal before it reaches the foreign object. Thirdly, most of the swab-like devices in the prior arts are of a straight elongated shaft and the user's hand will be blocking the line of vision of the user into the ear canal. Still, the size of the swab makes it difficult to have direct visualization of the ear canal. Furthermore, the swab-like devices do not provide features that safeguard against tremor of the user's hand.

Finally, Japanese Patent 2004194859 (as best as can be determined by available machine translation) apparently teaches a removal tool that has several disadvantages. The tool requires the use of a pair of tweezers to be advanced into the ear canal, adding complexity to the removal procedure. The tweezers, like the swab-like devices, also lacks features that minimize tremor of the user's hand. The tassel lacks rigidity and makes it inevitable that the adhesives on the tassel would make contact with the wall of the external ear canal. The adhesive applied on the tassel also requires additional curing time, making this invention impractical to be used with a child.

In summary, there is still a need for a device that allows for speedy and efficient removal of foreign bodies (or objects) stuck inside the external auditory canal, especially of a child patient, in an outpatient setting, where suction and irrigation tools are not readily available, and minimizes the chance of causing trauma to the auditory canal or the tympanic membrane when using the device.

### OVERVIEW

As set out in further detail below, an aspect of an embodiment of the present invention provides, among other things, a low-cost device (and related method) for removing foreign bodies (or objects) using an adhesive. An aspect of an embodiment of the present invention provides, but not limited thereto, a device comprising an elongated member having a first portion, a second portion with a distal end, and a fulcrum disposed along the elongated member between the first portion and the second portion. Furthermore, the device has a distinct bend, forming an offset angle between the first portion and the second portion. The distal end of the second portion comprises further a rounded tip and an adhesive portion. The device is intended to be inserted into the external ear canal and advanced incrementally until the foreign body (or object) adheres to the distal end of the second portion of the device. No curing time is required for the foreign body to adhere to the device. The device can then be withdrawn from the ear canal with the foreign body (object) attached to it. This device can be used by any medical professional in the Emergency Department (ED), a Pediatrics clinic, Family Medicine clinic, or an Otolaryngology clinic. The device may specialize in the extraction of non-graspable, spherical objects, and therefore provides advantages and unique features over conventional approaches. Accordingly, an embodiment of the present invention can be also be used in the removal of non-spherical objects, which gives it all-around utility.

In one embodiment, the device is of a small profile such that it can be inserted deeper than a swab-like device, and the tip of the device can reach the narrowing section of the external ear canal, the bony-cartilaginous junction, without making inadvertent contact with the wall of the external ear canal. In one embodiment, the tip of the device can reach the region of the external ear canal at the anterior tympanic sulcus, without making inadvertent contact with the wall of the external ear canal or with the tympanic membrane.

In one embodiment, the offset angle of the device allows visualization of the foreign body when it is being removed by the device. The device may be inserted into the ear canal with or without the aid of a speculum. The small profile and the distinct bend clear the path for a light source to provide better visualization of the foreign body while it is being removed by the device. The device may also be used in combination with an otoscope and a speculum if enhanced visualization is required.

In one embodiment of the present invention, the device has a finger support surface, such as a thumb loop (or solid thumb hold or thumb plate), which allows the practitioner to fulcrum the device against the thumb, as it is held between the thumb and the first three fingers, for instance. The finger support surface allows the practitioner to hold the device steadily to minimize tremor in the user. When held this way, the practitioner can also rest the hand against the patient's head further stabilizing the device and hand. Also, when held in this manner, if the patient or subject moves, the hand of the practitioner and device can move with the patient, minimizing traumatic injury to the ear canal. In an embodiment, the finger support surface also gives haptic feedback (tactile feedback) to the practitioner so he/she can “feel” when the end of the device makes contact with the foreign body. In an embodiment, the thumb loop, thumb hold or thumb plate allows better grip and balance of the device during the foreign body removal rather than attempting to grip the instrument at the other parts of the device.

In summary, an aspect of various embodiments of the present invention may provide a number of advantages, such as but not limited thereto, the following: i) the inexpensive cost to manufacture and maintain the device; ii) the device's special ability to extract spherical objects from the ear canal using an adhesive, making it available for use in the emergency room, family medicine, and pediatric offices, which do not have suction and other equipment; iii) the rounded tip of the device to minimize inadvertent damage to the wall of the external ear canal and to maximize the surface area of the adhesive; iv) the small profile of the device allowing it to go deeper than a swab-like device and also to prevent inadvertent contact of the device with the wall of the external ear canal; v) the small profile of the device to allow visualization of the foreign body being removed; vi) the device having a distinct bend or contour to get the user's hand out of the way to provide visualization of the foreign body; vii) a fulcrum with a finger support surface to stabilize the practitioner's hand and enabling the practitioner's hand to rest on the patient's head; vii) the finger support surface providing haptic feedback when it makes contact with the foreign body; viii) the flexibility of using the device with or without a speculum or an otoscope; and ix) the adhesive not requiring curing time and thus aiding in speedy removal of the foreign body.

An aspect of an embodiment of the present invention provides, but not limited thereto, a device for removing a foreign body from the external ear canal of a subject. The device may comprise: an elongated member having a first portion, a second portion with a distal end, and a fulcrum disposed along the elongated member between the first portion and the second portion; the first portion and the second portion configured with an offset angle so that the first portion and the second portion intersect at the fulcrum to provide better visualization of the external ear canal and better control of the device to minimize trauma to the external ear canal; the first portion configured to be held by a user with the user's fingers at least partially grasping at the fulcrum; and the distal end on the second portion having a rounded tip and an adhesive portion configured for attaching to and removing the foreign body from the external ear canal of the subject.

An aspect of an embodiment of the present invention provides, but not limited thereto, a method for removing a foreign body from the external ear canal of a subject. The method may comprise: providing an elongated member having a first portion and a second portion with a distal end; the distal end having an adhesive portion disposed thereon for attaching to and removing the foreign body from the external ear canal of the subject; holding the first portion of the elongated; inserting the distal end into the ear canal; contacting the foreign body with the adhesive portion to attach to the foreign body; and retrieving the elongated member from the ear canal with the foreign body attached thereto.

An aspect of an embodiment of the present invention provides, but not limited thereto, a device and related method of use for removing a foreign object from the external ear canal or the nasal cavity is provided. The device may comprise an elongated member having a first portion, a second portion with a distal end, and a fulcrum disposed along the elongated member between the first and the second portions. The first and second portions are configured with an offset angle so that the two portions interest at the fulcrum to provide better visualization of the external ear canal or the nasal cavity and better control of the device to minimize trauma to the ear canal or nasal cavity. The first portion is configured to be held by a user with the user's fingers at least partially grasping at the fulcrum, which may further comprise a thumb plate or loop. The distal end of the second portion has a rounded tip and an adhesive portion, which is inserted into the ear canal or the nasal cavity for attaching to and removing the foreign object when the distal end of the second portion is retrieved from the external ear canal or the nasal cavity.

These and other objects, along with advantages and features of various aspects of embodiments of the invention disclosed herein, will be made more apparent from the description, drawings and claims that follow.

### BRIEF DESCRIPTION OF THE DRAWINGS

The accompanying drawings, which are not necessarily drawn to scale, and which are incorporated into and form a part of the instant specification, illustrate several aspects and embodiments of the present invention and, together with the description therein, serve to explain the principles of the invention. The drawings are provided only for the purpose of illustrating select embodiments of the invention and are not to be construed as limiting the invention.

FIGS. 1A-1C provide perspective views of the invention in accordance with one embodiment of the present invention.

FIG. 1D provides a perspective view of another embodiment of the present invention as shown in FIGS. 1A-1C.

FIG. 1E provides a perspective view of another embodiment of the present invention as shown in FIGS. 1A-1C.

FIGS. 2A-2E provide perspective views of variations of the embodiments of the present invention as shown in FIGS. 1A-1E, respectively.

FIGS. 3A-3D provide perspective views of the device in accordance with another embodiment of the present invention.

FIG. 4A provides schematic illustration of one embodiment of the present invention inserted into an external ear canal of a subject with a foreign body.

FIG. 4B provides schematic illustration of a swab-like device inserted into an external ear canal of a subject with a foreign body.

FIG. 5A provides a schematic illustration of one embodiment of the present invention inserted through a speculum into an external ear canal of a subject with a foreign body.

FIG. 5B provides a schematic illustration of one embodiment of the present invention inserted through an otoscope used in combination with a speculum into an external ear canal of a subject with a foreign body.

FIGS. 6A-6B provide schematic illustrations of a distal end of a second portion of the device in accordance with various embodiments of the present invention.

### DETAILED DESCRIPTION OF EXEMPLARY EMBODIMENTS

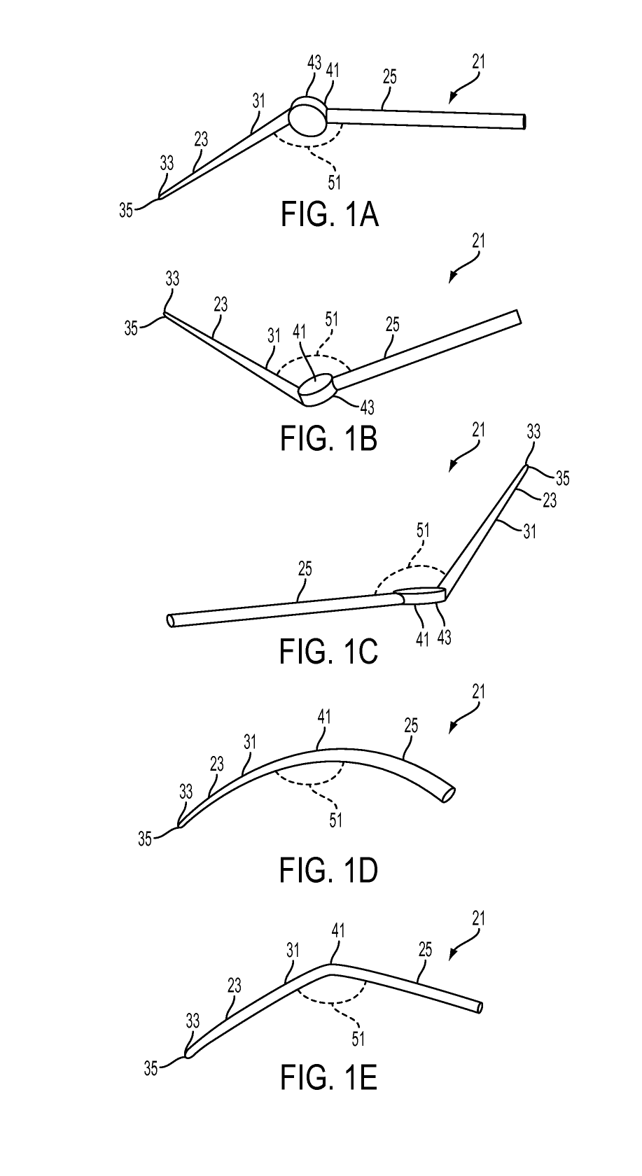

FIG. 1A provides the perspective view of the device 21 for removing of a foreign body 2 in the external ear canal 12 in accordance with one embodiment of the present invention. FIGS. 1B and 1C provide alternative perspective views of the device 21 as shown in FIG. 1A. The device 21 comprises an elongated member 23 having a first portion 25, a second portion 31 with a distal end 33, and a fulcrum 41 disposed along the elongated member 23 between the first portion 25 and the second portion 31. The first portion 25 and the second portion 31 are configured with an offset angle 51 between the first portion 25 and the second portion 31 so that the first portion 25 and the second portion 31 intersect at the fulcrum 41. In one embodiment of the present invention, the length along the longitudinal axis of the first portion 25 is approximately 70-120 mm. In one embodiment of the present invention, the length along the longitudinal axis of the second portion 31 is approximately 70-120 mm. It should be appreciated that the length of the first portion 25 and second portion 31 may be longer than 120 mm or shorter than 70 mm as desired or required. The first portion 25 is configured to be held by a user with the user's fingers at least partially grasping at a finger support surface 43 located at or near the fulcrum 41. In an embodiment, the finger support surface 43 may be a thumb plate or other type of surface or structure to be utilized by the user. It should be appreciated that the thumb plate or the like may coincide with fulcrum to optimize line of sight and haptic control, or may be offset from the fulcrum. In one embodiment of the present invention, the finger support surface 43 may be substantially round in shape and approximately 10-14 mm in diameter. It should be appreciated that the finger support surface 43 may be of any shape and various sizes and thicknesses. It should be appreciated that the finger support surface 43 may be greater than 14 mm or less than 10 mm as desired or required. In one embodiment of the present invention, the offset angle 51 is approximately 110-150 degrees. As set out in further detail below, the offset angle 51 and the dimension of the elongated member 23 are configured to provide better visualization of the external ear canal 12. The offset angle 51 also provides better control of the device 21 by allowing the user's hand to rest on the patient's head while using the device 21 to remove a foreign body 2 in the external ear canal 12. The user's hand can also move along with the patient's head if the patient's head moves while the device 21 is being removed, thereby minimizing trauma to the external ear canal 12. The offset angle 51 and finger support surface 43 also reduce tremor of the user's hand. It should be appreciated that the offset angle 51 may be greater than 150 degrees or less than 110 degrees as desired or required.

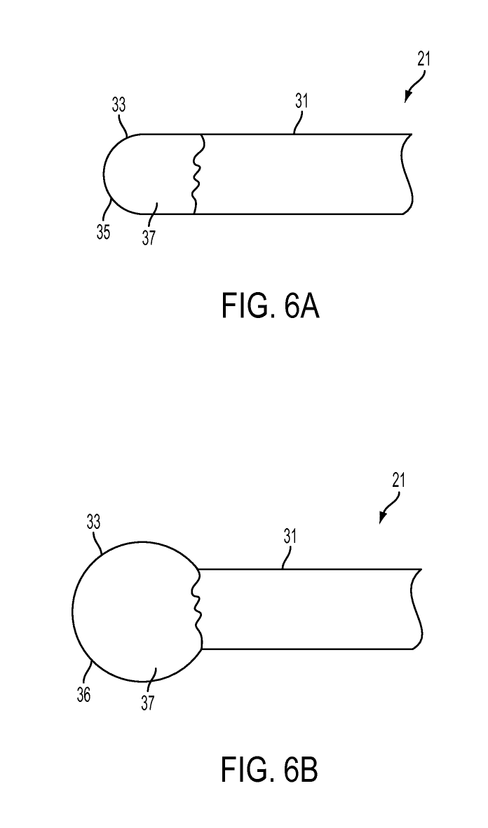

Still referring to FIGS. 1A-1C, the distal end 33 of the second portion 31 comprises further a rounded tip 35 and an adhesive portion 37 (as shown, for example, in FIG. 6A). Also as shown in FIG. 6A, which illustrates the distal end of the second portion of the device according to one embodiment of the present invention, the adhesive portion 37 may overlap with a significant portion of the rounded tip 35. It should be appreciated that the area of the adhesive portion 37 relative to areal of rounded tip 35 may vary relative to one another as desired or required. In an embodiment, the largest cross-sectional dimension of the rounded tip 35 may be in the range of about 1-5 mm. It should be appreciated that the cross-sectional dimension of the rounded tip 35 may vary as desired or required. The cross-sectional dimension of the rounded tip 35 may less than 1 mm or greater than 5 mm if desired or required. Absence of sharp edges on the rounded tip prevents the rounded tip 35 from damaging the wall of the external ear canal 12 if the device 21 makes inadvertent contact with the wall of the external ear canal 12. In one embodiment, the adhesive has been applied onto the adhesive portion before packaging so that the device 21 is ready for use. In another embodiment, the adhesive may be applied just prior to use by the user (or some combination of before packaging and at time of use).

Still referring to FIGS. 1A-1C, in one embodiment of the present invention, the elongated member 23 comprises a substantially circular cross-section. The diameter of the cross section is approximately 3-7 mm, with 5 mm being the preferred diameter for providing a sufficiently large adhesive portion 37 at the rounded tip 35 to contact the foreign body 2 and still leaving plenty of space in the external ear canal 12 for a good line of sight by the practitioner. It should be appreciated that the largest diameter of the cross-section may be greater than 7 mm or the smallest diameter may be less than 3 mm as desired or required. For example, in testing an embodiment of the present invention device, the elongated member 23 having a 5 mm diameter cross-section, the device provided superior line of sight for the user as well as sufficient structural integrity. Additionally, the 5 mm diameter cross-section had a corresponding rounded tip that provided ample surface to allow the adhesive to adhere to it. The 5 mm cross-section elongated member 23 and corresponding rounded tip demonstrated the fastest extraction times in anatomical model trials. Elongated members with a smaller cross section may suffer from being too flexible and lacking structural integrity, as well as insufficient surface area to accommodate adhesion. Whereas, elongated members with a larger cross section may suffer from restricting line of sight for the user and inadvertently hitting the walls of the ear canal as well as other areas.

It should be appreciated that the elongated member 23 may have cross-section that is circular or a variety of shapes, such as oval, rectangular, hexagonal or other polygonal shapes, as well as having irregular contours and multiple curvatures.

In one embodiment of the present invention, the diameter of the second portion 31 closer to the fulcrum 41 is larger than the diameter of the second portion 31 closer to the rounded tip 35 so that the second portion 31 comprises a tapered shaft. The largest diameter may be up to 5 mm and the smallest diameter may be as thin as 1 mm. It should be appreciated that the largest diameter may be greater than 5 mm or the smallest diameter may be less than 1 mm as desired or required.

An aspect of an embodiment of the present invention, as shown in FIG. 1D, provides a device 21 for removing of a foreign body 2 in the external ear canal 12. The device 21 comprises a curved elongated member 23 having a curved first portion 25, a curved second portion 31 with a distal end 33, and a fulcrum 41 disposed along the curved elongated member 23 between the first portion 25 and the second portion 31. The first portion 25 and the second portion 31 are configured with an offset angle 51 between the first portion 25 and the second portion 31 so that the first portion 25 and the second portion 31 intersect at the fulcrum 41. The offset angle 51 and the dimension of the elongated member 23 are configured to provide better visualization of the external ear canal 12. The offset angle 51 also provide better control of the device 21 by allowing the user's hand to rest on the patient's head while using the device 21 to remove a foreign body 2 in the external ear canal 12. The user's hand can move along with the patient's head if the patient's head moves while the device 21 is being removed, thereby minimizing trauma to the external ear canal 12. The distal end 33 of the second portion 31 comprises further a rounded tip 35 and an adhesive portion 37. In one embodiment, the elongated member 23 comprises a substantially circular cross-section. In one embodiment of the present invention as shown in FIG. 1D, the diameter of the second portion 31 closer to the fulcrum 41 is larger than the diameter of the second portion 31 closer to the rounded tip 35 so that the second portion 31 comprises a tapered shaft. It should be appreciated that the first and second portions of the elongated member may be configured to have different combinations of straight portions and curved portions.

FIG. 1E illustrated another embodiment of the present invention. The device 21 comprises an elongated member 23 having a straight first portion 25, a straight second portion 31 with a distal end 33, and a fulcrum 41 disposed along the elongated member 23 between the first portion 25 and the second portion 31. The first portion 25 and the second portion 31 are configured with an offset angle 51 between the first portion 25 and the second portion 31 so that the first portion 25 and the second portion 31 intersect at the fulcrum 41. The first portion 25 is configured to be held by a user with the user's fingers at least partially grasping at the fulcrum 41. As set out in further detail below, the offset angle 51 and the dimension of the elongated member 23 are configured to provide better visualization of the external ear canal 12. The offset angle 51 also provide better control of the device 21 by allowing the user's hand to rest on the patient's head while using the device 21 to remove a foreign body 2. The user's hand can move along with the patient's head if the patient's head moves while the device 21 is being removed, thereby minimizing trauma to the external ear canal 12. The distal end 33 of the second portion 31 comprises further a rounded tip 35 and an adhesive portion 37. In one embodiment of the present invention as shown in FIG. 1E, the elongated member 23 comprises a substantially uniform cross-section.

It should be appreciated that the cross-section of the elongated member may vary at different locations disposed along the axis of the elongated member 23 as desired or required.

In an alternative embodiment, the device 21 may have a first portion 25 that is pivotally or movably connected to the second portion 31 at the fulcrum 41 (or other desired location of connection) for relative movement between a collapsed configuration and a deployed position (either fully deployed position or one or more partially deployed positions). For instance, the device 21 may have a first portion 25 that is pivotally or movably connected to the second portion 31 at the fulcrum 41 for relative movement between a collapsed configuration and a deployed position in an embodiment in which the first portion and second portion lie substantially side by side (or other desired arrangement) and a fully deployed configuration in which said first portion and said second portion form the offset angle.

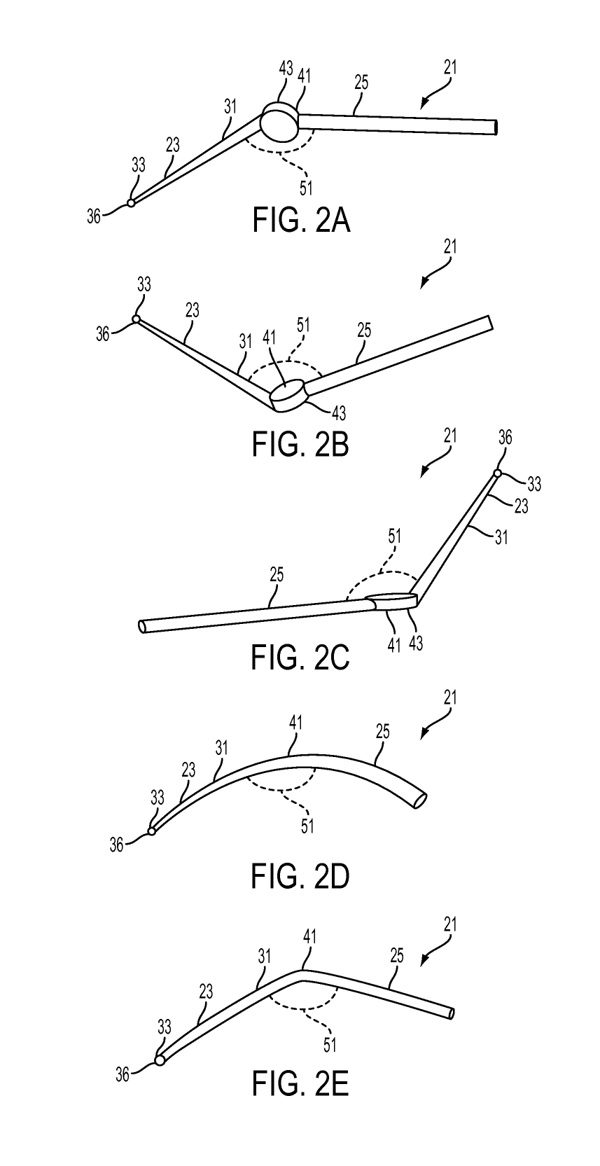

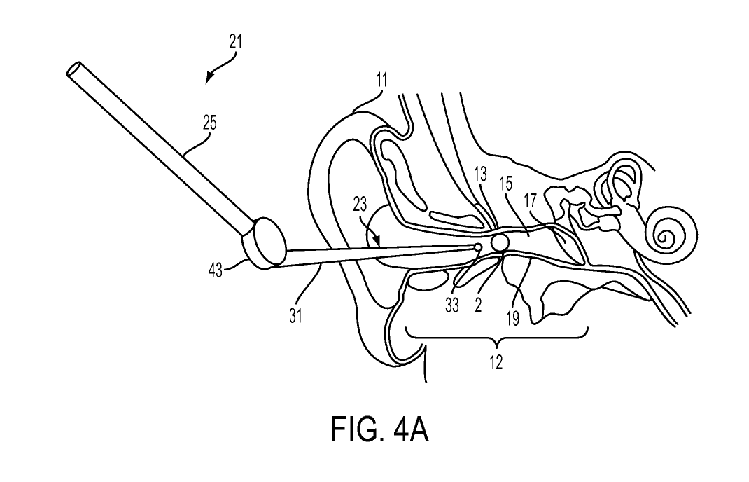

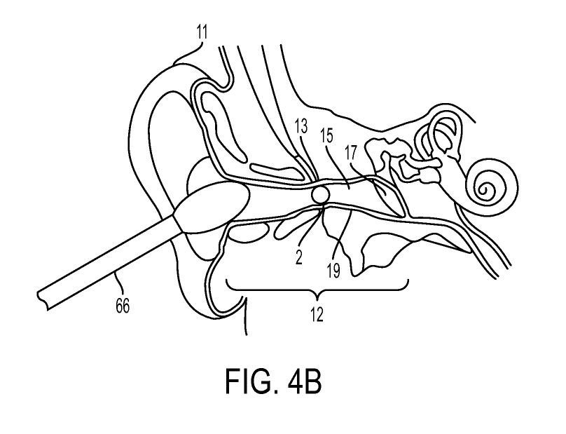

FIGS. 2A-2E provide perspective views of variations of the embodiments of the present invention as shown in FIGS. 1A-1E, respectively. An aspect of an embodiment of the present invention has the advantage of, but not limited thereto, providing a large contact surface between the adhesive portion 37 (as shown in FIG. 6B) and the foreign body 2 (as shown in FIG. 4A). As shown in FIGS. 2A-2E and FIG. 6B, the rounded tip may be in a substantially spherical shape 36. The adhesive portion 37, as shown on FIG. 6B, may overlap substantially with the substantially spherical shape 36 of the rounded tip 35. It should be appreciated that the diameter of the substantially spherical shape 36 may be smaller than, substantially the same as, or bigger than the diameter or the largest cross-sectional dimension of the distal end 33. In one embodiment of the present invention, the diameter of the substantially spherical 36 of the rounded tip is approximately 1-5 mm. It should be appreciated that the cross-sectional dimension of the rounded tip 35 may vary as desired or required. The cross-sectional dimension of the rounded tip 35 may less than 1 mm or greater than 5 mm if desired or required.

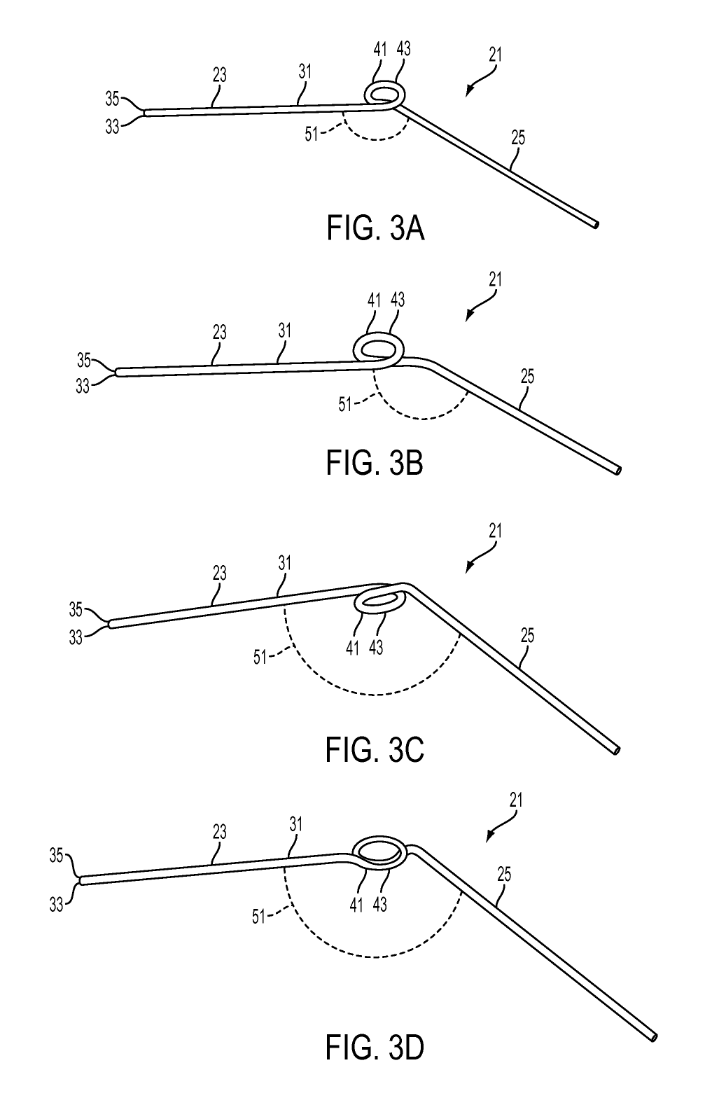

An aspect of a various embodiments of the present invention, as illustrated by FIGS. 3A-3D, provides a device 21 for removing of a foreign body 2 in the external ear canal 12 (as shown in FIG. 4A). The device 21 comprises an elongated member 23 having a first portion 25, a second portion 31 with a distal end 33, and a fulcrum 41 disposed along the elongated member 23 between the first portion 25 and the second portion 31. The first portion 25 and the second portion 31 are configured with an offset angle 51 between the first portion 25 and the second portion 31 so that the first portion 25 and the second portion 31 intersect at or adjacent to the fulcrum 41. The first portion 25 is configured to be held by a user with the user's fingers at least partially grasping at a finger support surface 43 located at the fulcrum 41. In one embodiment, as shown in FIGS. 3A-3D, the finger support surface 43 may be a centrally located loop for thumb control, formed with four bends of a 304 stainless steel wire. It should be appreciated that the device may be a wide variety of materials, such as, but not limited thereto, metals, alloys, plastics or polymers. FIGS. 3A-3D illustrate four different, non-limiting, examples to make the four bends to demonstrate the relative position of the center of the loop for thumb control and the fulcrum. Still referring to FIGS. 3A-3D, the distal end 33 of the second portion 31 comprises further a rounded tip 35 and an adhesive portion 37. Also as shown in FIG. 6A, which illustrates the distal end 33 of the second portion 31 of the device 21 according to one embodiment of the present invention, the adhesive portion 37 may overlap with a significant portion of the rounded tip 35. The largest cross-sectional dimension of the rounded tip 35, in accordance with one embodiment of the present invention, may be in the range of about 1-5 mm. In one embodiment, the adhesive has been applied onto the adhesive portion before packaging so that the device 21 is ready for use. In another embodiment, the adhesive may be applied just prior to use by the practitioner. Still referring to FIGS. 3A-3D, in one embodiment of the present invention, the elongated member 23 comprises a substantially circular cross-section. In one embodiment of the present invention, although not shown, the diameter of the second portion 31 closer to the fulcrum 41 may be larger than the diameter of the second portion 31 closer to the rounded tip 35 so that the second portion 31 comprises a tapered shaft.

Although not shown in the figures, it should be appreciated that the elongated member 23 of an embodiment of this present invention may be straight, or may have multiple curvatures along the elongated member 23. It should be appreciated that various sizes, dimensions, contours, rigidity, shapes, flexibility and materials of any of the components or portions of components in the various embodiments discussed throughout may be varied and utilized as desired or required—such as by taking on all shapes along the entire continual geometric spectrum of manipulation of x, y and z planes to provide and meet the anatomical and structural demands, operational and requirements.

Suitable materials and structures, for example, may vary as well. For example, the device may be made of a 304 stainless steel wire, which has four bends and a centrally located loop for thumb control, like the embodiment of the present invention as shown in FIGS. 3A-3D. The device 21 may also be made of plastic, such as the embodiment shown in FIGS. 1A-1E and FIGS. 2A-2E, bent at a similar angle, with a solid finger support surface 43 instead of a loop. The device may also be made of a silicone-like material, which has sufficient rigidity for the second portion to be inserted into the ear canal, and at the same time deformable when the rounded or substantially spherical tip comes into contact with the foreign body so as to increase contact surface between the adhesive on the tip and the foreign body. The adhesive applied to the tip of the device may be standard ‘super high tack’ GlueDots or the like, as well as other suitable adhesive materials or structures. It should be appreciated that in the case of adhesive failure, the adhesive portion 37 detaches from the foreign object 2 instead of detaching from the distal end 31 of the device 21, ensuring that the adhesive will not be lost in the ear canal. It should also be appreciated that, regardless of the rigidity of the elongated member 23 due to material or structure, the adhesive portion 37 itself may have a consistency that makes it slightly deformable when in contact with the foreign body 2 to increase contact surface between the adhesive portion 37 and the foreign body 2.

An aspect of an embodiment of the present invention, as illustrated in FIG. 4A, provide a device 21 to be used without the aid of other instruments, for removal of a foreign body 2 in the external ear canal 12. While the user is holding the ear 11 of the patient, the user's other hand will be hold the first portion 25 of the elongated member 23 of the device 21. The device 21 can be advanced incrementally towards the foreign body 2, which is stuck at the narrowing section, the bony cartilaginous junction 13. It should be appreciated that an advantage of this invention is, but not limited thereto, that the device 21 has a smaller profile than a swab-like device 66 (FIG. 4B) so that it can reach deeper than a swab-like device 66, as shown in FIGS. 4A and 4B. The diameter of a human's external ear canal is typically about 5 mm, and smaller for a child. The largest cross-sectional dimension of a commercially available Q-tip, which is a typical swab-like device, is about 6-7 mm. In contrast, an aspect of an embodiment of the present invention provides a device 21 for foreign body removal from the external ear canal 12, wherein the largest cross-sectional dimension of the second portion does not exceed about 5 mm. It should be appreciated that the cross-sectional dimension may be larger than 5 mm if desired or required. Similarly, it should be appreciated that the cross-sectional dimension may be smaller than 5 mm if desired or required. As a result, the swab-like device 66 as shown in FIG. 4B would not be able to reach a foreign body 2 stuck at the bony cartilaginous junction 13 or a foreign body 2 stuck further down at the osseous portion of the ear canal 15. Whereas, an advantage associated with an embodiment of the present invention device is that it would be able to reach a foreign body 2 stuck at the bony cartilaginous junction 13 or a foreign body 2 stuck further down at the osseous portion of the ear canal 15.

Another advantage of an embodiment of the present invention over a swab-like device 66 is that the distal end 33 of the device 21 will not tear when the device 21 is pulled out together with the foreign body 2, whereas the cotton or sponge material on the tip of the swab-like device may tear and be lost in the ear canal 12. Another advantage of an embodiment of the present invention over forceps is that the device 21 will be particularly good at removing non-graspable or spherical bodies or objects, such as beads, popcorn kernels, beans, vegetable matter, pebbles, concrete sediments (or sediments of other material), and various plastic, metal, ceramic, glass, wood, or polymer pieces or chips, or the like. It should be appreciated that any solid or firm object that fits in the external ear canal may be retrieved with an embodiment of the present invention device.

It should also be appreciated that an embodiment of the present invention device (and related method) may also be used to remove cerumen (dead skin and secretions) and other ear debris formed in the external ear canal, such as desquamated epithelium (dead skin) and hair, or even for dirt. Although not shown, the ear debris may be generally located on the canal wall or suspended in the canal similar to the location shown as the foreign body.

Another advantage of an embodiment of the present invention over devices terminating in a hook or a loop is that the device 21 does not have to advance beyond the foreign body 2 in the tight space of the external ear canal 12, where it is often impossible for the hook to advance beyond the foreign body 2. Although not shown in FIG. 4A, it should be appreciated that the dimension of an embodiment of the present invention device 21 is that it may be used in the situation when the foreign body 2 is small enough to pass the bony cartilaginous junction 13 and fall due to gravity towards the anterior sulcus 19. An embodiment of the present invention device 21 may be small and long enough to reach into the osseous portion of ear canal 15 to be next to the tympanic membrane 17.

Still referring to FIG. 4A, it should be appreciated that the offset angle 51 between the first portion 25 and the second portion 31 of an embodiment of the present invention device allows the user's hand to be out of the line of vision into the external ear canal 12. In addition, the small size of the second portion 31 of the device 21 also provides direct visualization of the foreign body 2. It should be appreciated that a viewing light, not shown in FIG. 4A, can be used to enhance visualization into the ear canal 12. It should also be appreciated that the finger support surface 43 of the device 21, which the user's thumb will at least be partially grasping, provides haptic feedback when the distal end 33 of the second portion 31 of the device 21 makes contact with the foreign body 2.

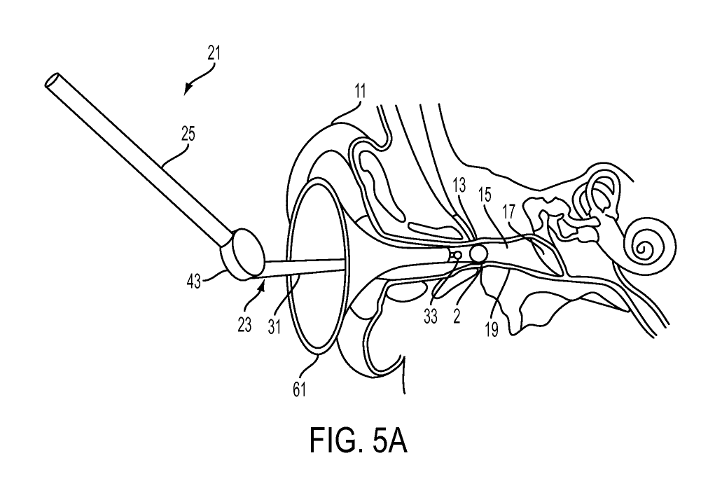

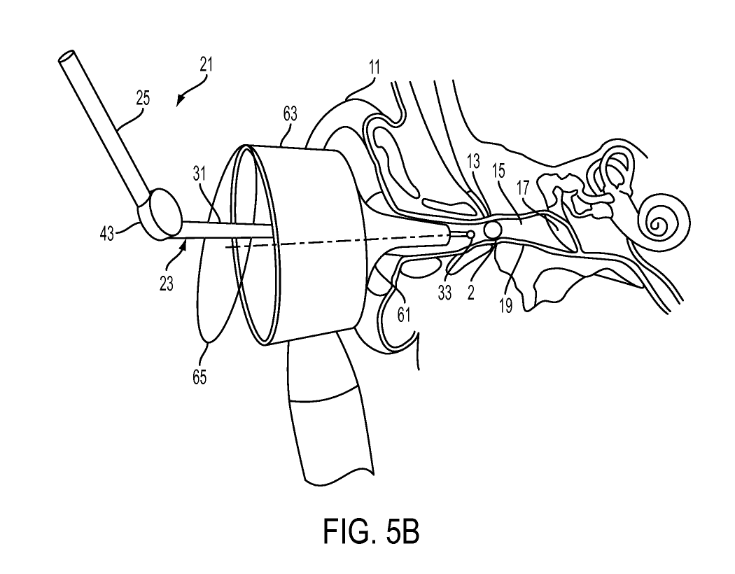

FIG. 5A is a schematic illustration of an aspect of an embodiment of the present invention providing a device 21 used in combination with a speculum 61 for removal of a foreign body 2 in the external ear canal 12. The speculum 61 may be reusable or of single-use. For example, reusable standard speculums with a 4 mm diameter are readily available and disposable speculums of a larger than 4 mm diameter are also available. It should be appreciated that the diameter may be smaller or greater than 4 mm for either type. The user first fits the speculum 61 into the ear canal 12 while grabbing on the pinna of the ear 11, and then advances the second portion 31 of the device 21 via the throughbore of the speculum 61 into the ear canal 12. It should be appreciated that a viewing light, not shown in FIG. 5A, can be used to enhance visualization into the ear canal 12 via the throughbore of the speculum 61. After the adhesive portion 37 of the device 21 makes contact with the foreign body 2, giving a haptic feedback to the user via the finger support surface 43, the device 21 may be retracted with the foreign body 2 adhered onto it via the throughbore of the speculum 61. It should be appreciated that after the adhesive portion 37 of the device 21 makes contact with the foreign body 2, the device 21 and the speculum 61 may be retracted simultaneously if the foreign body 2 is too large to be removed via the throughbore of the speculum 61.

FIG. 5B is a schematic illustration of an aspect of an embodiment of the present invention providing a device 21 used in combination with a speculum 61 and an otoscope 63 for removal of a foreign body 2 in the external ear canal 12. The otoscope 63 is fitted onto the speculum 61 before the user first fits the speculum 61 into the ear canal 12 while grabbing on the pinna of the ear 11. The user then slightly displaces lens 65 on the otoscope 63 to advance the second portion 31 of the device 21 via the throughbores of the otoscope 63 and the speculum 61 into the external canal 12. It should be appreciated that a viewing light, not shown in FIG. 5B, can be used to enhance visualization into the ear canal 12. After the adhesive portion 37 of the device 21 makes contact with the foreign body 2, giving a haptic feedback to the user's hand on the finger support surface 43, the device 21 may be retracted with the foreign body 2 via the throughbores of the otoscope 63 and the speculum 61. It should be appreciated that after the adhesive portion 37 of the device 12 makes contact with the foreign body 2, the device 21, the otoscope 63 and the speculum 61 may be retracted simultaneously if the foreign body 2 is too large to be removed via the throughbore of the speculum 61. It should also be appreciated that instead of displacing the lens of the otoscope to advance the an embodiment of the present invention into the ear canal, an otoscope having a modified lens with at least one aperture that accommodates the entry of an embodiment of the present invention may be used in the place of a standard otoscope. It should be appreciated that other devices, tools and instruments (or modifications thereof) may be implemented with the various approaches of the present invention device and related method of use.

Various components of the device may be reusable and/or disposable after each use. For instance, in one embodiment, the adhesive component or material of the device may need to be replaced after each use. Accordingly, an embodiment of the present invention device is designed to be manufactured as one piece; however it can also be manufactured in separated pieces and assembled prior to use. For example, it should be appreciated that the second portion may be detachable from the device at the fulcrum after each use and a new second portion may be assembled onto the device at the fulcrum, by snap fit, adhesives, or other methods apparent to those skilled in the art. In another example, although not shown in the figures, it should be appreciated that the device may be foldable at or near the fulcrum and be unfolded into a fully deployed configuration before use.

Accordingly, an embodiment of the present invention device is designed to remove foreign bodies from a child's ear canal (or subjects or patients of any age); however, it can also be used to remove a foreign body from other anatomical spaces. It should be appreciated that an embodiment of the present invention device (and related method) may be used to remove foreign bodies from the nose. It should also be appreciated that an embodiment of the present invention device (and related method) may also be used to remove ear debris, including cerumen or the like in the external ear canal.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example. It should be appreciated that the dimensions provided for the device in the present disclosure are associated with humans—particularly the ear canal. It should be appreciated that the scale of the dimension of the device may vary relative to the given species, animal type, or subject size. It should be appreciated that the scale of the dimension of the device may vary if utilized for other anatomical spaces, such as the nasal cavity.

It should be appreciated that various sizes, dimensions, contours, rigidity, shapes, flexibility and materials of any of the components or portions of components in the various embodiments discussed throughout may be varied and utilized as desired or required. Similarly, locations and alignments of the various components may vary as desired or required.

It should be appreciated that any of the components or modules referred to with regards to any of the present invention embodiments discussed herein, may be integrally or separately formed with one another. Further, redundant functions or structures of the components or modules may be implemented.

It should be appreciated that the device and related components discussed herein may take on all shapes along the entire continual geometric spectrum of manipulation of x, y and z planes to provide and meet the anatomical, environmental, and structural demands and operational requirements. Moreover, locations and alignments of the various components may vary as desired or required.

### EXAMPLES

Practice of an aspect of an embodiment (or embodiments) of the invention will be still more fully understood from the following example, which is presented herein for illustration only and should not be construed as limiting the invention in any way.

### Example Set No. 1

TABLE 1
Comparative Analysis. Cost to manufacture an
embodiment of the present invention device as
compared to the retail cost of closest competitors.

| Cost to Manufacture Our Device | Retail Cost of Competitors |
|---|---|
| Metal Prototype-$0.17 | $26-381 (Alligator Forceps) |
| Plastic Prototype-$4.95 | $50 (Katz Extractor) |
| | $80 (Irrigation System) |
| | $60 (Suction device) |

### Example Set No. 2

For the purpose of prototype testing of an embodiment of the present invention, the present inventors narrowed the maximum weight that the adhesive could hold for 12 seconds (approximately equal to the average extraction time for the slowest user in our time trial test, plus one standard deviation). The adhesive was capable of holding 40 grams for 12 seconds without deforming (stretching), and 140 g for 12 seconds with moderate deformation. These masses are orders of magnitude greater than the mass of an average bead or popcorn kernel, so the adhesive demonstrated strength and reliability far beyond its normal operating range.

### Additional Examples

### Example 1

An aspect of an embodiment provides, but not limited thereto, a device for removing a foreign body from the external ear canal [or nasal cavity] of a subject. The device may comprise: an elongated member having a first portion, a second portion with a distal end, and a fulcrum disposed along the elongated member between the first portion and the second portion; the first portion and the second portion configured with an offset angle so that the first portion and the second portion intersect at the fulcrum to provide better visualization of the external ear canal [or nasal cavity] and better control of the device to minimize trauma to the external ear canal [or nasal cavity]; the first portion configured to be held by a user with the user's fingers at least partially grasping at the fulcrum; and the distal end on the second portion having a rounded tip and an adhesive portion configured for attaching to and removing the foreign body from the external ear canal [or nasal cavity] of the subject.

### Example 2

The device of example 1, wherein said rounded tip comprises a substantially spherical shape.

### Example 3

The device of example 1 (as well as subject matter of example 2), wherein said fulcrum comprises an enlarged portion for ease of holding with fingers.

### Example 4

The device of example 1 (as well as subject matter of one or more of any combination of examples 2-3), wherein said second portion has a largest cross-sectional dimension that allows said second portion to advance to the narrowest section of the external ear canal [or nasal cavity].

### Example 5

The device of example 1 (as well as subject matter of one or more of any combination of examples 2-4), wherein said second portion has a largest cross-sectional dimension that allows said second portion to advance to the bone-cartilaginous junction of the external ear canal.

### Example 6

The device of example 1 (as well as subject matter of one or more of any combination of examples 2-5), wherein said second portion has a largest cross-sectional dimension that allows said second portion to reach the region of the external ear canal at the anterior tympanic sulcus.

### Example 7

The device of example 1 (as well as subject matter of one or more of any combination of examples 2-6), wherein said second portion has a largest cross-sectional dimension that allows said second portion to reach the region of the external ear canal adjacent to the tympanic membrane.

### Example 8

The device of example 1 (as well as subject matter of one or more of any combination of examples 2-7), wherein said second portion has a largest cross-sectional dimension that allows said second portion to advance to the narrowest section of the external ear canal [or nasal cavity] without making contact with the wall of the external ear canal [or nasal cavity].

### Example 9

The device of example 1 (as well as subject matter of one or more of any combination of examples 2-8), wherein said second portion has a largest cross-sectional dimension that allows said second portion to reach the region of the external ear canal at the anterior tympanic sulcus without making contact with the wall of the external ear canal.

### Example 10

The device of example 1 (as well as subject matter of one or more of any combination of examples 2-9), wherein said second portion comprises a tapered shaft with the cross-sectional dimension of said tapered shaft being larger closer to said fulcrum than the cross-sectional dimension of said tapered shaft closer to said rounded tip.

### Example 11

The device of example 1 (as well as subject matter of one or more of any combination of examples 2-10), wherein said rounded tip is deformable to increase contact area with the foreign body.

### Example 12

The device of example 1 (as well as subject matter of one or more of any combination of examples 2-11), wherein said adhesive portion is deformable to increase contact area with the foreign body.

### Example 13

The device of example 1 (as well as subject matter of one or more of any combination of examples 2-12), wherein said second portion is long enough and contoured to be used with a speculum and said rounded tip and said second portion are small enough to pass through the speculum.

### Example 14

The device of example 13 (as well as subject matter of one or more of any combination of examples 2-12), wherein said rounded tip and said second portion are small enough to pass through a partially displaced lens of an otoscope so that said device can be used in combination with a speculum and an otoscope.

### Example 15

The device of example 1 (as well as subject matter of one or more of any combination of examples 2-14), wherein said adhesive portion does not require cure time.

### Example 16

The device of example 1 (as well as subject matter of one or more of any combination of examples 2-15), wherein said device provides haptic feedback when said device is in contact with the foreign body in the external ear canal [or nasal cavity].

### Example 17

The device of example 1 (as well as subject matter of one or more of any combination of examples 2-16), wherein said device is made of a metal.

### Example 18

The device of example 1 (as well as subject matter of one or more of any combination of examples 2-17), wherein said device is made of plastic.

### Example 19

The device of example 2 (as well as subject matter of one or more of any combination of examples 3-18), wherein said substantially spherical shaped tip is deformable to increase contact area with the foreign body.

### Example 20

The device of example 2 (as well as subject matter of one or more of any combination of examples 3-19), wherein said adhesive portion is deformable to increase contact area with the foreign body.

### Example 21

The device of example 2 (as well as subject matter of one or more of any combination of examples 3-20), wherein said second portion is long enough and contoured to be used with a speculum and said substantially spherical shaped rounded tip and said second portion are small enough to pass through said speculum.

### Example 22

The device of example 1 (as well as subject matter of one or more of any combination of examples 2-21), wherein the control reduces tremor by the user.

### Example 23

The device of example 1 (as well as subject matter of one or more of any combination of examples 2-22), wherein the control improves maneuverability by the user.

### Example 24

The device of example 13 (as well as subject matter of one or more of any combination of examples 2-12 and 14-23), further comprising said speculum provided together in a kit with said device.

### Example 25

The device of example 14 (as well as subject matter of one or more of any combination of examples 2-13 and 15-24), further comprising said speculum and said otoscope provided together in a kit with said device.

### Example 26

The device of example 1 (as well as subject matter of one or more of any combination of examples 2-25), wherein said foreign body includes at least one or more of the following: bead, popcorn kernel, bean, vegetable matter, pebble, concrete sediment, or a plastic, metal, ceramic, glass, wood, or polymer piece or chip.

### Example 27

The device of example 1 (as well as subject matter of one or more of any combination of examples 2-26), wherein said adhesive portion is configured for contacting and removing ear debris [or nasal debris] that is located in the external ear canal [or nasal cavity] of the subject from the external ear canal [or nasal cavity].

### Example 28

The device of example 1 (as well as subject matter of one or more of any combination of examples 2-27), wherein said first portion is pivotally connected to said second portion at said fulcrum for relative movement between a collapsed configuration and a deployed configuration, wherein said first portion and said second portion form said offset angle.

### Example 29

The device of example 3 (as well as subject matter of one or more of any combination of examples 2 and 4-28), wherein said enlarged portion comprises at least one of the following: thumb loop, thumb hold, or thumb plate.

### Example 30

An aspect of an embodiment provides, but not limited thereto, a method for removing a foreign body from the external ear canal [or nasal cavity] of a subject. The method may comprise: providing an elongated member having a first portion and a second portion with a distal end; the distal end having an adhesive portion disposed thereon for attaching to and removing the foreign body from the external ear canal [or nasal cavity] of the subject; holding the first portion of the elongated; inserting the distal end into the ear canal [or nasal cavity]; contacting the foreign body with the adhesive portion to attach to the foreign body; and retrieving the elongated member from the ear canal [or nasal cavity] with the foreign body attached thereto.

### Example 31

The method of example 30, further comprising:

providing a speculum; and

passing said distal end through a speculum.

### Example 32

The method of example 31, further comprising:

providing an otoscope; and

passing said distal end through a partially displaced lens of an otoscope.

### Example 33

The method of example 30 (as well as subject matter of one or more of any combination of examples 31 and 32), further comprising:

contacting ear debris located in the external canal with said adhesive portion so as to attach to the ear debris; and

retrieving said elongated member from the ear canal [or nasal cavity] with the ear debris [or nasal debris] attached thereto.

### Example 34

The method of using any of the devices or its components provided in any one or more of examples 1-29.

### Example 35

The method of manufacturing any of the devices or its components provided in any one or more of examples 1-29.

### REFERENCES

The following patents, applications and publications as listed below and throughout this document are hereby incorporated by reference in their entirety herein. The devices, systems, compositions, and methods of various embodiments of the invention disclosed herein may utilize aspects disclosed in the following references, applications, publications and patents and which are hereby incorporated by reference herein in their entirety (and which are not admitted to be prior art with respect to the present invention by inclusion in this section):
A. U.S. Pat. No. 5,454,817, Katz, D., “Oto-Nasal Foreign Body Extractor”, Oct. 3, 1995.
B. U.S. Pat. No. 8,551,031, Edme, et al., “Swab Applicator and Methods of Use”, Oct. 8, 2013.
C. U.S. Pat. No. 5,158,532, Peng, et al., “Articulated Swab”, Oct. 27, 1992.
D. U.S. Pat. No. 5,133,721, Angulo, “Device for Removing Foreign Objects from Anatomic Organs”, Jul. 28, 1992.
E. U.S. Pat. No. 6,316,540, Gauderer, et al., “Apparatus for Removal of Esophageal Coins and Similarly Shaped Objects”, Mar. 26, 2002.
F. U.S. Patent Application Publication No. US 2012/0203256 A1, Huttner, et al., “Method of Removing Cerumen or a Foreign Body from an Ear Canal and Articulating Curette for use Therewith”, Aug. 9, 2012.
G. U.S. Patent Application Publication No. US 2010/0121363 A1, Huttner, et al., “Articulating Curette”, May 13, 2010.
H. U.S. Patent Application Publication No. US 2008/0208100 A1, Wolff, R., “Method and Apparatus for Removal of Cerumen”, Aug. 28, 2008.
I. International Patent Application Serial No. PCT/US2007/020395, entitled “Tube, Stent and Collar Insertion Device”, filled Sep. 20, 2007.
J. Japanese Patent No. 2004194859 (A), Yoshito, “Foreign Matter Removal Tool”, Jul. 16, 2004.
K. International Patent Application Serial No. PCT/US2006/017906, entitled “Surgical Tool and Insertion Device for Tube-Placement”, filed May 5, 2006.

### WORKS CITED

L. Schulze S L, Kerschner J, Beste D, Pediatric external auditory canal foreign bodies: a review of 698 cases. Otolarngol Head Nick Surg 2002; 127: 73-78.
M. DiMuzio J, Deschler D, Emergency Department Management of Foreign Bodies of the External Ear Canal in Children, Otol & Neurotol 2002; 23: 473-5.
N. Kadish H, Ear and Nose Foreign Bodies: “It is All About the Tools,” Clin Pediatr 2005; 44: 665-70.
O. D. L. Katz, Oto-nasal foreign body extractor, U.S. Pat. No. 5,454,817, 1995.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims (or disclosure herein), including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

We claim:

1. A device for removing a foreign body from the external ear canal of a subject, said device comprising:
an elongated member having a first portion, a second portion with a distal end, and a fulcrum disposed along said elongated member between said first portion and said second portion; said first portion and said second portion configured with an offset angle so that said first portion and said second portion intersect at said fulcrum to provide better visualization of the external ear canal and better control of said device to minimize trauma to the external ear canal;
said first portion configured to be held by a user with said user's fingers at least partially grasping at said fulcrum; and
said distal end on said second portion having a rounded tip and an adhesive portion configured for attaching to and removing the foreign body from the external ear canal of the subject.

2. The device of claim 1, wherein said rounded tip comprises a substantially spherical shape.

3. The device of claim 1, wherein said fulcrum comprises an enlarged portion for ease of holding with fingers.

4. The device of claim 1, wherein said second portion has a largest cross-sectional dimension that allows said second portion to advance to the narrowest section of the external ear canal.

5. The device of claim 1, wherein said second portion has a largest cross-sectional dimension that allows said second portion to advance to the bone-cartilaginous junction of the external ear canal.

6. The device of claim 1, wherein said second portion has a largest cross-sectional dimension that allows said second portion to reach the region of the external ear canal at the anterior tympanic sulcus.

7. The device of claim 1, wherein said second portion has a largest cross-sectional dimension that allows said second portion to reach the region of the external ear canal adjacent to the tympanic membrane.

8. The device of claim 1, wherein said second portion has a largest cross-sectional dimension that allows said second portion to advance to the narrowest section of the external ear canal without making contact with the wall of the external ear canal.

9. The device of claim 1, wherein said second portion has a largest cross-sectional dimension that allows said second portion to reach the region of the external ear canal at the anterior tympanic sulcus without making contact with the wall of the external ear canal.

10. The device of claim 1, wherein said second portion comprises a tapered shaft with the cross-sectional dimension of said tapered shaft being larger closer to said fulcrum than the cross-sectional dimension of said tapered shaft closer to said rounded tip.

11. The device of claim 1, wherein said rounded tip is deformable to increase contact area with the foreign body.

12. The device of claim 1, wherein said adhesive portion is deformable to increase contact area with the foreign body.

13. The device of claim 1, wherein said second portion is long enough and contoured to be used with a speculum and said rounded tip and said second portion are small enough to pass through the speculum.

14. The device of claim 13, wherein said rounded tip and said second portion are small enough to pass through a partially displaced lens of an otoscope so that said device can be used in combination with a speculum and an otoscope.

15. The device of claim 1, wherein said adhesive portion does not require cure time.

16. The device of claim 1, wherein said device provides haptic feedback when said device is in contact with the foreign body in the external ear canal.

17. The device of claim 1, wherein said device is made of a metal.

18. The device of claim 1, wherein said device is made of plastic.

19. The device of claim 2, wherein said substantially spherical shaped tip is deformable to increase contact area with the foreign body.

20. The device of claim 2, wherein said adhesive portion is deformable to increase contact area with the foreign body.

21. The device of claim 2, wherein said second portion is long enough and contoured to be used with a speculum and said substantially spherical shaped rounded tip and said second portion are small enough to pass through said speculum.

22. The device of claim 1, wherein the control reduces tremor by the user.

23. The device of claim 1, wherein the control improves maneuverability by the user.

24. The device of claim 13, further comprising said speculum provided together in a kit with said device.

25. The device of claim 14, further comprising said speculum and said otoscope provided together in a kit with said device.

26. The device of claim 1, wherein said foreign body includes at least one or more of the following: bead, popcorn kernel, bean, vegetable matter, pebble, concrete sediment, or a plastic, metal, ceramic, glass, wood, or polymer piece or chip.

27. The device of claim 1, wherein said adhesive portion is configured for contacting and removing ear debris that is located in the external ear canal of the subject from the external ear canal.

28. The device of claim 1, wherein said first portion is pivotally connected to said second portion at said fulcrum for relative movement between a collapsed configuration and a deployed configuration, wherein said first portion and said second portion form said offset angle.

29. The device of claim 3, wherein said enlarged portion comprises at least one of the following: thumb loop, thumb hold, or thumb plate.

30. A method for removing a foreign body from the external ear canal of a subject, said method comprising:
providing an elongated member having a first portion and a second portion with a distal end;
said distal end having an adhesive portion disposed thereon for attaching to and removing the foreign body from the external ear canal of the subject;
holding said first portion of said elongated;
inserting said distal end into the ear canal;
contacting said foreign body with said adhesive portion to attach to the foreign body; and
retrieving said elongated member from the ear canal with the foreign body attached thereto.

31. The method of claim 30, further comprising:
providing a speculum; and
passing said distal end through a speculum.

32. The method of claim 31, further comprising:
providing an otoscope; and
passing said distal end through a partially displaced lens of an otoscope.

33. The method of claim 30, further comprising:
contacting ear debris located in the external canal with said adhesive portion so as to attach to the ear debris; and
retrieving said elongated member from the ear canal with the ear debris attached thereto.
